(19) United States
(12) Patent Application Publication
(10) Pub. No.: US 2009/0187156 A1
(43) Pub. Date: Jul. 23, 2009

(54) Diaper comprising adjustable and sealable leg openings

(76) Inventor: Melanie K. Anzalone, Omaha, NE (US)

Correspondence Address: Benjamin I. Edlavitch, PO Box 220661, Chicago, IL, 60622-0661, US

(21) Appl. No.: 12/315,746

(22) Filed: Dec. 5, 2008

Related U.S. Application Data
(63) Continuation of application No. 12/009,211, filed on Jan. 17, 2008, now abandoned.

(51) Int. Cl. A61F 13/15

(52) U.S. Cl. 604/385.03; 604/385.25; 604/385.24; 604/390; 604/389; 604/385.01

(57) ABSTRACT

A diaper comprising adjustable leg openings, which inhibit leakage through the leg openings. The diaper may be a pull-on type diaper or a sheet structure comprising a front waist portion and rear waist portion that attach to each other in a manner that allows the sheet structure to form a pair of leg openings and a waist opening. The diaper may comprise an elastic structure adjacent to or abutting the leg openings and the waist opening. In any variation of the diaper the periphery of the leg openings are adjustable. Furthermore, the diaper may include strips that seal the leg openings to a thigh of a person wearing the diaper.

### CROSS REFERENCE TO RELATED APPLICATION

This application is a continuation of application Ser. No. 12/009,211, filed Jan. 17, 2008, the entire contents of which are hereby incorporated by reference.

### BACKGROUND

The present disclosure relates to diapers. The present disclosure also relates to disposable diapers of the type that are worn by infants, adults, and incontinent people.

A great number of diapers and disposable diapers have been brought to market. One type of diaper is a flat type that the consumer can fold and fasten into an underwear-like structure. Another popular type of diaper is manufactured and sold to consumers in a prefabricated underwear-like structure (i.e. pull-on type diaper).

Several implementations of both basic types of diaper comprise an elastic structure that is adjacent to or abutting each leg opening of a diaper. The elastic structure is meant to allow the periphery of the leg opening to fit snuggly against the thigh of a person wearing the diaper. Unfortunately, for many people that wear a diaper, the leg opening does not fit snugly against the thigh of that person.

Furthermore, in cases where the leg opening snugly fits against a person's thigh, the elastic structures are often not tight enough or secure enough to prevent leakage from the leg opening, especially leakage of heavier feces.

### SUMMARY

The present application discloses a diaper comprising adjustable leg openings that assist in the prevention of leakage through the leg openings of the diaper.

The diaper may be a pull-on type diaper or a sheet structure comprising a front waist portion and rear waist portion that attach to each other in a manner that allows the sheet structure to form a pair of leg openings and a waist opening. The diaper may comprise an elastic structure adjacent to or abutting the leg openings and the waist opening.

A leg opening may be adjusted by an adhesive strip or flap attached to a portion of the periphery of the leg opening, which has the ability to attach to another portion of the periphery of a leg opening allowing for the leg opening to tighten. On the other hand, the leg openings may be adjusted by a tightening band structure that encircles the periphery of a leg opening or another means for tightening a leg opening known to those of ordinary skill in the art.

In addition, preferred embodiments of the diaper include a strip that seals the leg opening of the diaper to a thigh of a person wearing the diaper.

### BRIEF DESCRIPTION OF THE DRAWINGS

Below is a description of example embodiments of a diaper comprising adjustable leg openings in conjunction with appended drawings, wherein like reference numerals refer to like elements in figures, and wherein:

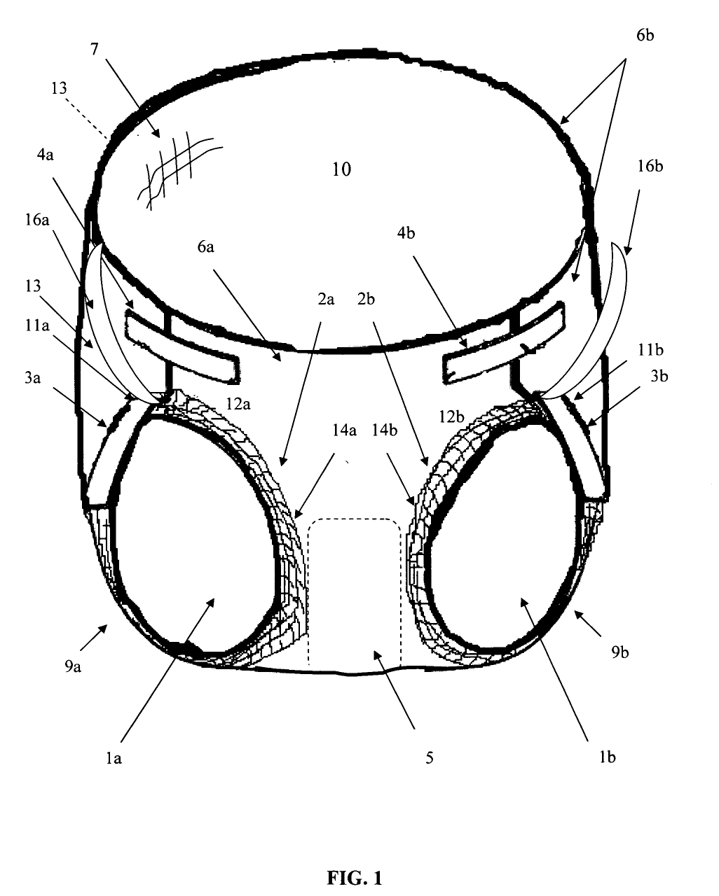

figure one (labeled “FIG. 1” in the drawings) is a drawing of a preferred embodiment of the diaper;

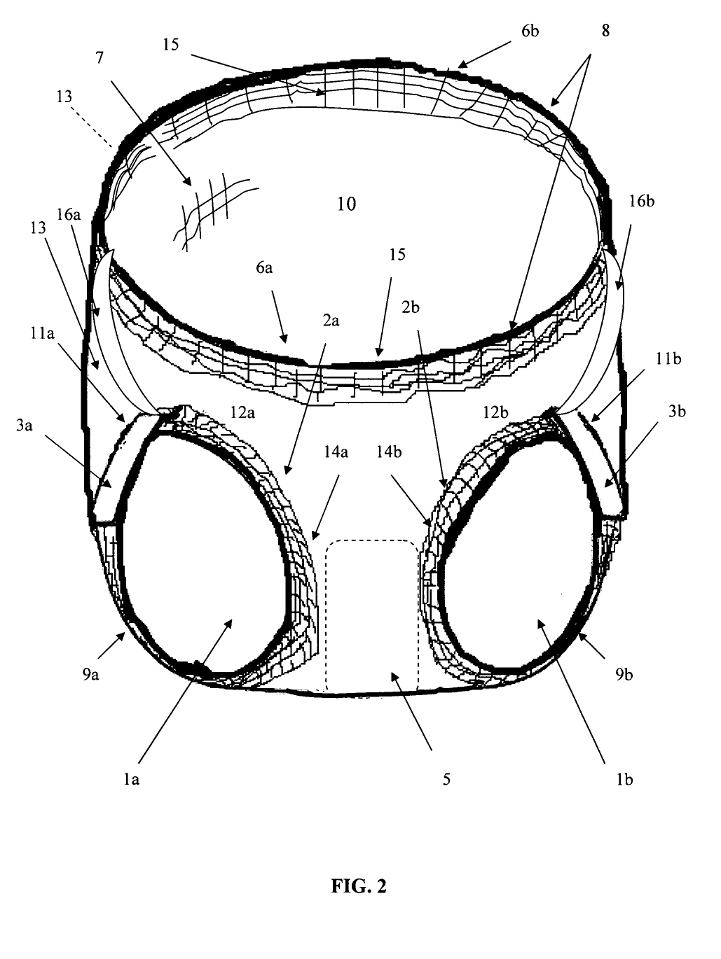

figure two (labeled “FIG. 2” in the drawings) is a drawing of a preferred pull-up type embodiment of the diaper;

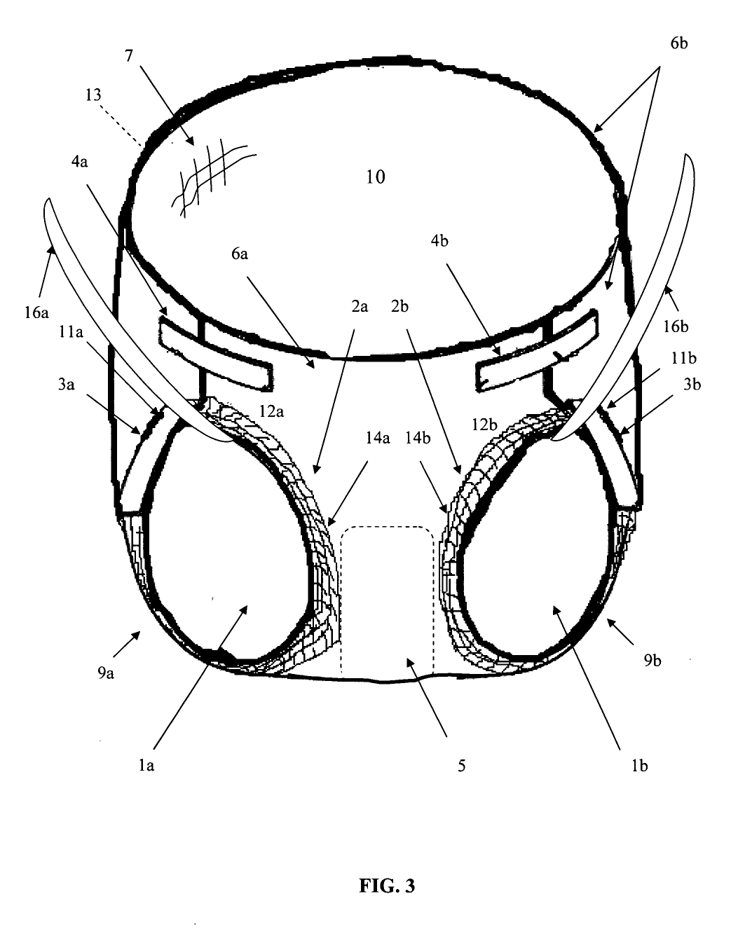

figure three (labeled “FIG. 3” in the drawings) is a drawing of a preferred embodiment of the diaper, where a leg opening constriction mechanism 3a & 3b and a sealing strip 16a & 16b abut the boundary of each leg opening of the diaper;

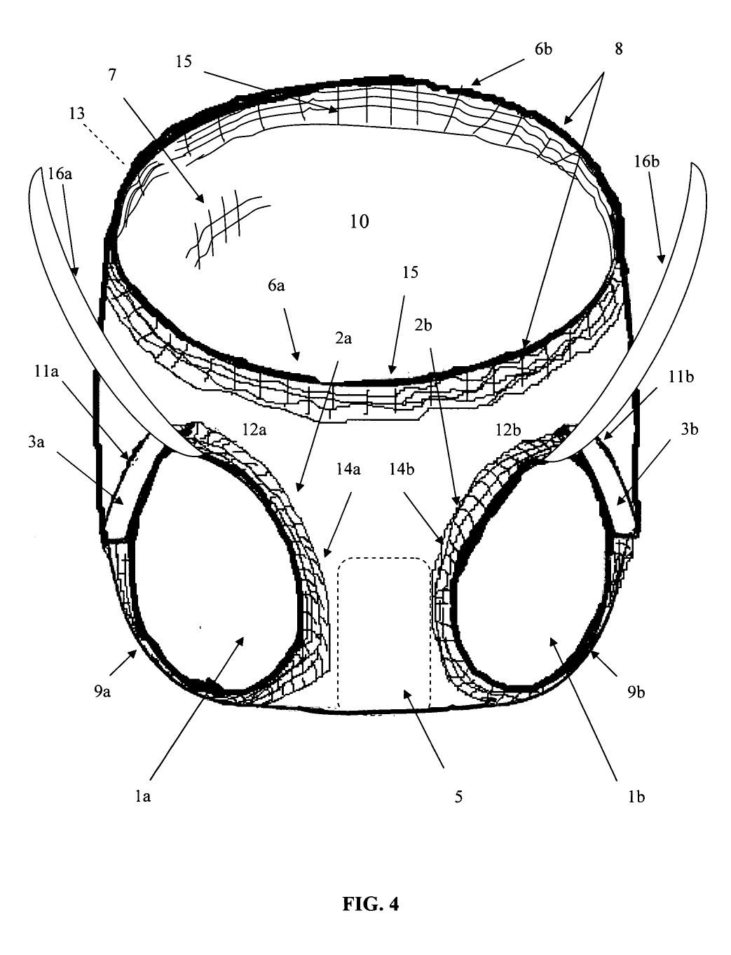

figure four (labeled “FIG. 4” in the drawings) is a drawing of a preferred pull-up type embodiment of the diaper, where a leg opening constriction mechanism 3a & 3b and a sealing strip 16a & 16b abut the boundary of each leg opening of the diaper;

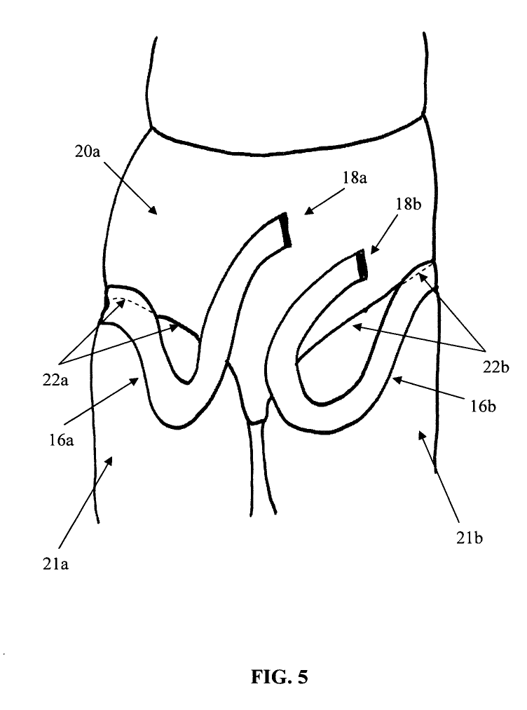

figure five (labeled “FIG. 5” in the drawings) is a front-view drawing of a preferred embodiment of the diaper, where the diaper is on a person;

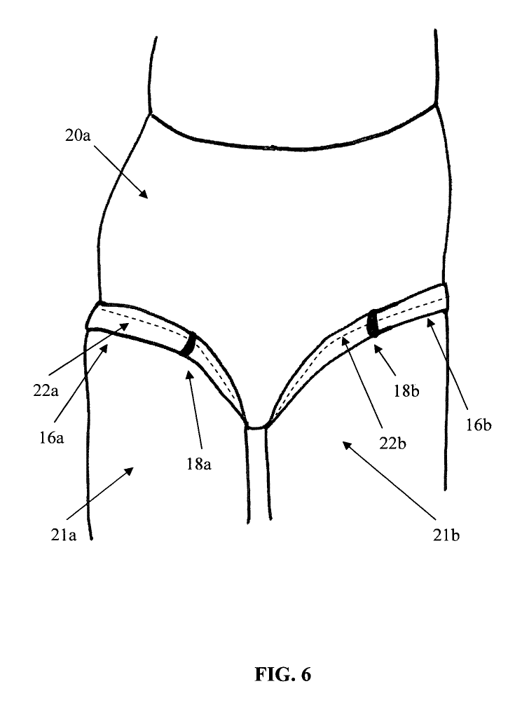

figure six (labeled “FIG. 6” in the drawings) is a front-view drawing of a preferred embodiment of the diaper, where the diaper is on a person and a sealing strip 16a & 16b wraps around the boundary 22a & 22b of each leg opening and adheres to the diaper and the thigh 21a & 21b of the person; and

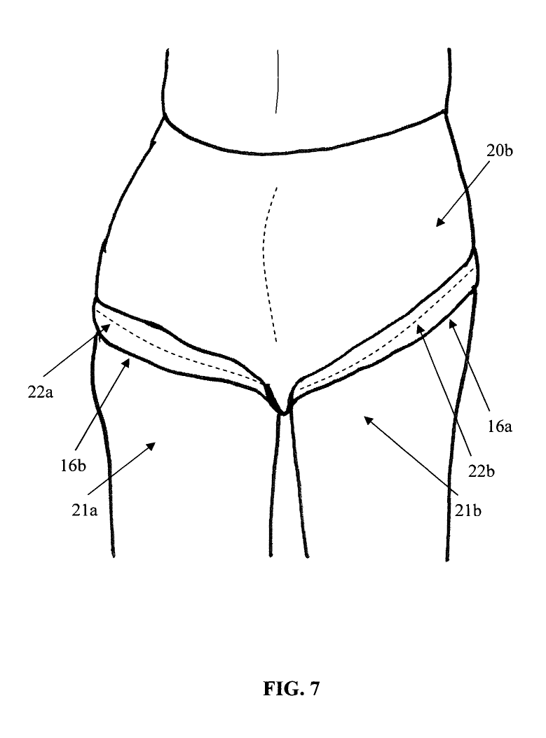

figure seven (labeled “FIG. 7” in the drawings) is a rear-view drawing of a preferred embodiment of the diaper, where the diaper is on a person and a sealing strip 16a & 16b wraps around the boundary 22a & 22b of each leg opening and adheres to the diaper and the thigh 21a & 21b of the person.

### DETAILED DESCRIPTION OF THE EMBODIMENT

The present specification explains preferred embodiments of a diaper comprising adjustable leg openings. The present detailed description does not limit the diaper and the diaper may embody various forms. Furthermore, figures one and two illustrate preferred embodiments of the diaper and the figures are merely devices for assisting the explanation of the present detailed description and do not limit the scope of the diaper. In addition, the figures are not drawn to scale.

In general, the disclosure embodies all diapers, disposable and non-disposable diapers, which have adjustable leg openings.

As provided by figure one, a preferred embodiment of the diaper comprises a sheet structure comprising a front waist portion 6a and rear waist portion 6b, and first and second side portions 9a & 9b. Each side portion 9a or 9b comprises a concave portion 2a & 2b. The front waist portion 6a and the rear waist portion 6b each comprise an attachment mechanism 4a & 4b that allows the front waist portion 6a to attach to the back waist portion 6b in a manner that allows the sheet structure to form a pair of leg openings 1a & 1b and a waist opening 10. The leg openings 1a & 1b are derived from the curling of the concave portions 2a & 2b of the side portions 9a & 9b of the diaper. As previously mentioned, the front and rear waist portions 6a & 6b combine into the waist opening 10, when the periphery of the front waist portion 6a and the back waist portion 6b attach to each other by adhesive flaps (i.e. attachment mechanisms) 4a & 4b.

As shown in figures one and two, unique to other diapers, the diaper disclosed in this application comprises a leg opening constriction mechanism 3a & 3b, which allows adjusting the size of each leg opening 1a & 1b. Furthermore, the diaper includes a strip 16a & 16b that seals the leg opening 1a & 1b of the diaper to a thigh of a person wearing the diaper. One end of the strip 16a & 16b adheres to the leg opening constriction mechanism 3a & 3b (as shown in figures one and two) or a portion of the diaper adjacent to or abutting the leg opening. The remainder of the strip not attached to the diaper comprises at least one adhesive surface that adheres to the skin of a person. The length of the strip 16a & 16b is long enough to wrap around the thigh of a person and the leg opening creating a tight seal of the leg opening to the thigh.

In preferred embodiments of the diaper, the seal is airtight.

As shown in figure six, in preferred embodiments of the diaper, the front end 18a & 18b of the strip 16a & 16b after wrapping around the entire boundary 22a & 22b of the leg opening is in the front of the diaper for easy adjustment.

Figure five, six, and seven illustrate the two strips 16a & 16b on the diaper that are long enough to wrap around the thighs of a person 21a & 21b and the leg openings creating a tight seal of the leg openings to the thighs. In figure five, the two strips are not wrapped around the thighs and leg openings. In figure six, the two strips 16a & 16b are wrapped around the thigh and leg openings. The strips 16a & 16b cover each boundary 22a & 22b of the leg openings. By covering each boundary 22a & 22b of leg openings, the strip 16a & 16b further prevents leakage out of the leg openings.

In figures five and six, the figures illustrate the front-side of the diaper 20a. Figure seven illustrates the backside of the diaper 20b.

In preferred embodiments of the diaper, the adhesive surface comprises an adhesive that allows the strip to be peeled away from the skin of a person with minimum irritation to the skin of the person.

In other preferred embodiments of the diaper, as shown in figure two, the diaper may be a pull-up type diaper. A pull-up type embodiment, as show in figure two, comprises a sheet structure that comprises a front waist portion 6a, a rear waist portion 6b, and a first and second side portion 9a & 9b. The front waist portion 6a and the rear waist portion 6b define a waist opening 10. Each of the side portions 9a & 9b have a concave portion 2a & 2b defining leg openings 1a & 1b; and each of the leg openings 1a & 1b comprise a mechanism 3a & 3b that allows adjusting the size of each leg opening 1a & 1b.

In figures one and two, the leg openings 1a & 1b may be constricted by pulling the attached leg opening flaps (i.e. leg opening constriction mechanism) 3a & 3b towards the front of the diaper and adhering a front portion 11a & 11b of the flaps to a front portion 12a & 12b of the diaper. In other embodiments of the diaper, the leg openings may be constricted by pulling the attached leg opening flaps towards the back of the diaper and adhering a front portion of the flaps to a back portion of the diaper.

In other embodiments of the diaper, the leg openings may be adjusted by a tightening band structure that encircles the periphery of a leg opening or another means for tightening a leg opening known to those of ordinary skill in the art.

As previously mentioned, the diaper may include a strip 16a & 16b that seals the leg opening 1a & 1b of the diaper to a thigh of a person wearing the diaper. One end of the strip 16a & 16b adheres to the leg opening constriction mechanism 3a & 3b (as shown in figures one and two) or a portion of the diaper adjacent or abutting the leg opening. The remainder of the strip not attached to the diaper includes at least one adhesive surface that adheres to the skin of a person, which allows the strip to wrap around the thigh of a person and the leg opening creating a seal of the leg opening to the thigh. It is preferred that the adhesive surface comprises an adhesive that allows the strip to be peeled away from the skin of a person with minimum irritation to the skin of the person.

Where a strip or a flap comprises an adhesive it may be preferred to have an additional strip attached to the adhesive portion to prevent wear of the adhesive prior to use. Where an additional strip adheres to the adhesive portion of a strip or a flap, the additional strip should have a surface that allows easy removal from the adhesive strip or flap.

In preferred embodiments of the diaper, as demonstrated in figure one and two, the sheet structure comprises an inner sheet 7, an outer sheet 13, and an absorbent member 5; and the absorbent member 5 lies between the inner sheet 7 and the outer sheet 13.

In preferred embodiments of the diaper, as demonstrated in figure one and two the inner sheet 7 is liquid permeable, and the outer sheet 13 is liquid impermeable.

As previously mentioned, and as shown in figures one and two, the diaper further comprises elastic structures 14a & 14b adjacent to or abutting each of the concave portions 2a & 2b of the side portions 9a & 9b of the diaper; and the elastic structures 14a & 1 4b are adapted to form a gather.

As shown in figure two, a preferred embodiment of the diaper further comprises an elastic structure 15 adjacent to the waist opening; and the elastic structure 15 is adapted to form a gather.

Not shown in the drawings, preferred embodiments of the diaper comprise at lease one easy to rip portion. The easy to rip portion may be any easy to rip structure known to those of ordinary skill in the art. For example, the rip structure may be an array of perforations in the diaper. In more preferred embodiments, the easy to rip portion extends from a top edge of the waist opening to a top edge of the leg opening.

1. A diaper comprising in combination:
a sheet structure comprising a front waist portion, a rear waist portion, a first side portion, and a second side portion;
each said side portion comprises a concave portion;
said front waist portion and said rear waist portion each comprise an attachment mechanism that allows said front waist portion to attach to said back waist portion in a manner that allows said sheet structure to form two leg openings, where a periphery of each said leg opening is defined by said concave portion, and a waist opening, where a periphery of said waist opening is a combination of said front waist portion and said back waist portion; and
abutting each said leg opening said diaper comprises a mechanism that allows adjusting of each said leg opening.

2. Said diaper of claim 1, further comprising a strip, where an end of said strip adheres to a portion of said diaper abutting said leg opening and a remaining portion of said strip comprises at least one adhesive surface that adheres to skin of a person.

3. Said diaper of claim 2, wherein said adhesive surface that adheres to skin of a person comprises an adhesive that allows said strip to be peeled away from skin of a person with minimum irritation to said skin.

4. Said diaper of claim 1, wherein said sheet structure comprises an inner sheet, an outer sheet, and an absorbent member; and said absorbent member resides between said inner sheet and said outer sheet.

5. Said diaper of claim 4, wherein said inner sheet is liquid permeable, and said outer sheet is liquid impermeable.

6. Said diaper of claim 1, further comprising an elastic structure adjacent to each said concave portion of said side portions; and said elastic structure is adapted to form a gather.

7. Said diaper of claim 1, further comprising at least one easy to rip portion.

8. Said diaper of claim 7, wherein said easy to rip portion extends from a top edge of said waist opening to a top edge of said leg opening.

9. A pull-on type diaper comprising in combination:
a sheet structure comprising a front waist portion, a rear waist portion, a first side portion, and a second side portion;
said front waist portion and said rear waist portion define a waist opening;
each said side portion comprises a concave portion that defines two leg openings; and
adjacent to each said leg opening said diaper comprises a mechanism that allows adjusting size of each said leg opening.

10. Said diaper of claim 9, further comprising a strip, where an end of said strip adheres to a portion of said diaper abutting said leg opening and a remaining portion of said strip comprises at least one adhesive surface that adheres to skin of a person.

11. Said diaper of claim 3, wherein said adhesive surface that adheres to skin of a person comprises an adhesive that allows said strip to be peeled away from skin of a person with minimum irritation to said skin.

12. Said diaper of claim 9, wherein said sheet structure comprises an inner sheet, an outer sheet, and an absorbent member; and said absorbent member resides between said inner sheet and said outer sheet.

13. Said diaper of claim 12, wherein said inner sheet is liquid permeable, and said outer sheet is liquid impermeable.

14. Said diaper of claim 9, further comprising an elastic structure adjacent to each said concave portion of said side portions; and said elastic structure is adapted to form a gather.

15. Said diaper of claim 9, further comprising an elastic structure adjacent to said waist opening; and said elastic structure is adapted to form a gather.

16. Said diaper of claim 9, further comprising at least one easy to rip portion.

17. Said diaper of claim 16, wherein said easy to rip portion extends from a top edge of said waist opening to a top edge of said leg opening.
